(19) United States
(12) Patent Application Publication
(10) Pub. No.: US 2024/0265995 A1
(43) Pub. Date: Aug. 8, 2024

(54) System of Predicting Sensitivity of Klebsiella against Cefoxitin and Method

(71) Applicants: Peking Union Medical College Hospital, Chinese Academy of Medical Sciences, Beijing (CN); Hangzhou Jieyi Biotechnology Company Limited, Hanzhou City (CN)

(72) Inventors: Qiwen Yang, Beijing (CN); Jing CHEN, Beijing (CN); Wei YU, Beijing (CN); Jun WANG, Beijing (CN); Xiaobing CHU, Beijing (CN); Shiyu CHEN, Beijing (CN); Qian ZHANG, Beijing (CN); Yao YAO, Beijing (CN)

(21) Appl. No.: 18/523,709

(22) Filed: Nov. 29, 2023

(30) Foreign Application Priority Data
Feb. 6, 2023 (CN) 202310065401.4

(51) Int. Cl. G16B 20/10

(52) U.S. Cl. CPC G16B 20/10; G16B 40/00; G16B 50/10; G16B 50/30

(57) ABSTRACT

Disclosed are a system and method of predicting sensitivity of Klebsiella against Cefoxitin, which belong to bioinformatics art. The system comprises a computer readable storage medium on which is stored a computer program. An Exp (−k) power value calculation method is implemented when the computer program is executed by a processor. The Exp(−k) power value calculation method comprises following computing steps: S1: k value is calculated according to formula I:

$$k = 0.032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) \qquad Formula I$$

S2: Exp(−k) power value with natural constant e as base and −k as exponent is calculated; wherein, C1-C5 are respectively the number of ramA, sul1, KPC-1, DHA-1, bleomycin resistance determinant gene copies in the candidate Klebsiella strain. The accuracy of predicting sensitivity of Klebsiella against Cefoxitin using the prediction method and prediction system of the present invention is about 94.7%.

### CROSS-REFERENCE TO RELATED APPLICATION

This application claims the benefit of and priority to Chinese patent application 202310065401.4 filed Feb. 6, 2023, which is incorporated herein by reference in its entirety.

### TECHNICAL FIELD

This present invention belongs to bioinformatics art, specifically refers to a system and method of predicting sensitivity of Klebsiella against Cefoxitin.

### BACKGROUND

Antibiotics were once a “secret weapon” for humans to fight against many diseases. In the late 19th and early 20th centuries, the discovery of a series of antibiotics greatly increased human lifespan. In recent years, with the continuous application of antibiotics, drug abuse has gradually emerged, which leads to an increase in clinical antibiotic resistance and adverse reactions, and brings a heavy burden to the global economy. Effectively controlling the abuse of antibiotics in healthcare is an important link in addressing the global issue of antibiotic resistance.

Pathogenic microorganisms refer to microorganisms that can invade the human body, and cause infections or even infectious diseases, which are also known as pathogens. Pathogenic microorganisms mainly include bacteria, viruses, fungi, parasites, mycoplasma, chlamydia, rickettsia, spirochetes, etc. There are various types of microbial samples. Intestinal samples include feces, mucous membranes, etc. Liquid samples include urine, blood, cerebrospinal fluid, saliva, sputum, alveolar lavage fluid, amniotic fluid, etc. Swab samples include samples from oral cavity, reproductive tract, skin, etc. Other samples include tissues, liver, eyes, placenta, etc.

The Latin scientific name of Genus Klebsiella is Klebsiella Trevisan, and its systematic classification level is Genus. It is a straight bacterium with a diameter of 0.3-1.0 μm, 0.6-6.0 in length μM, and it's single, paired, or short chain arrangement. Currently reported strains of this genus include Klebsiella pneumoniae, Klebsiella aerogenes, Klebsiella oxytoca, Klebsiella quasipneumoniae, Klebsiella variicola, Klebsiella michiganensis, etc.

Among them, Klebsiella pneumoniae, as a model strain of the genus Genus Klebsiella, is widely present in the environment, and is a common opportunistic pathogen that easily colonizes the respiratory and intestinal tracts of patients, and causes infections in multiple parts of the digestive tract, respiratory tract, blood, etc. It is one of the pathogenic bacteria that cause human pneumonia and is also one of the common drug-resistant bacteria in hospitals. According to a study by the Second Military Medical University, the resistance rate against meropenem of carbapenem resistant Klebsiella pneumoniae isolated from 2014 to 2017 to was 62.5% (252/403).

Cefoxitin is a cephalosporin antibiotic. It is a new type of antibiotic manufactured from Cephamycin C produced by Streptomyces Lactamdurans, through semi synthesis. Its mother nucleus is similar to cephalosporins, with similar antibacterial properties, and is customarily included in the second-generation cephalosporins class. Cefoxitin inhibits bacterial cell wall biosynthesis by binding to one or more penicillin binding proteins (PBPs), thereby exerting antibacterial effects on Gram negative, Gram positive, and anaerobic bacteria.

Bacterial drug sensitivity testing is currently the most commonly used method for detecting bacterial resistance in clinic and laboratory both domestically and internationally. There are methods such as paper disc method, agar dilution method, broth dilution method, and concentration gradient method. Except for the paper disc method, all other methods can obtain relatively accurate minimum inhibitory concentration (MIC) of drugs. It's required firstly to obtain pure culture in the bacterial drug sensitivity test, which is not suitable for difficult to cultivate and non-cultured bacteria, and takes a long time. Sometimes, it is difficult to meet the current clinical needs for rapid diagnosis and targeted treatment of severe and emergency infections. The traditional detection and identification methods of pathogenic microorganisms fail to meet the comprehensive needs of wide coverage, speed, and accuracy. The diagnosis and treatment of infectious diseases are mainly based on empirical and directional methods. Clinical doctors and patients urgently need innovative detection methods to identify infectious pathogens more comprehensively, accurately, and quickly, which can assist in diagnosis and reasonable standardized medication treatment, shorten treatment courses, reduce mortality rates, and reduce medical costs.

With the promotion of emerging technologies such as PCR technology, whole genome sequencing technology, microfluidic technology, VITEK-2 compact fully automated bacterial identification/drug sensitivity system, the exploration of new technologies for bacterial resistance detection is gradually deepening, and various new technologies and methods for bacterial resistance detection are becoming increasingly mature. Although the VITEK-2 compact fully automated bacterial identification/drug sensitivity system is simple and fast, its accuracy in bacterial identification/drug sensitivity evaluation is influenced by the sample status and bacterial culture, and its usage cost is relatively high.

Therefore, there is an urgent need to develop a system and method in this field that can quickly, accurately, and cost-effectively predict the sensitivity of Klebsiella strains against Cefoxitin.

### SUMMARY

In response to the aforementioned shortcomings and requirements of prior art in this field, the present invention aims to provide a system and method of predicting sensitivity of Klebsiella against Cefoxitin.

Technical solution of the present invention is as follows:

A system of predicting sensitivity of Klebsiella against Cefoxitin, comprises a computing unit. The computing unit comprises a computer readable storage medium on which is stored a computer program. An Exp (−k) power value calculation method is implemented when the computer program is executed by a processor. The Exp(−k) power value calculation method comprises following computing steps:

S1: k value is calculated according to formula I:

$$k = 0.032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) \qquad Formula I$$

$$k = 0. 032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} )$$

S2: Exp(−k) power value with natural constant e as base and −k as exponent is calculated;
in formula I,
C1 is the number of ramA gene copies in a candidate Klebsiella strain,
C2 is the number of sul1 gene copies in a candidate Klebsiella strain,
C3 is the number of KPC-1 gene copies in a candidate Klebsiella strain,
C4 is the number of DHA-1 gene copies in a candidate Klebsiella strain,
C5 is the number of bleomycin resistance determinant gene copies in a candidate Klebsiella strain.

In above formula I, the first pair of parentheses includes C1 minus 1.008 divided by 0.317; the second pair of parentheses includes C2 minus 0.667 divided by 1.326; the third pair of parentheses includes C3 minus 0.552 divided by 1.121; the fourth pair of parentheses includes C4 minus 0.072 divided by 0.377; and the fifth pair of parentheses includes C5 minus 0.036 divided by 0.292.

In above formula I, k=0.032-0.557×(C1 minus 1.008 divided by 0.317)+0.054×(C2 minus 0.667 divided by 1.326)+2.878×(C3 minus 0.552 divided by 1.121)+1.021×(C4 minus 0.072 divided by 0.377)+0.772×(C5 minus 0.036 divided by 0.292).

The system of predicting sensitivity of Klebsiella against Cefoxitin, also comprises a result output unit. The computing unit transmits the calculated Exp(−k) power value to the result output unit. The result output unit recognizes Exp(−k) power value and outputs result;

preferably, said natural constant e=2.718281828459045.
the result output unit outputs resistant result R when recognizing Exp(−k) power value<1;
the result output unit outputs sensitive result S when recognizing Exp(−k) power value≥1;
the result output unit and the computing unit are communicated through data-path, Exp(−k) power value calculated by the computing unit is transmitted to the result output unit through data-path;
preferably, said sensitive result S refers to that the candidate Klebsiella strain is sensitive to Cefoxitin, and said resistant result R refers to that the candidate Klebsiella strain is resistant against Cefoxitin.

The system of predicting sensitivity of Klebsiella against Cefoxitin, also comprises an experiment unit and a data input unit. The experiment unit and data input unit are communicated through a data-path. The experiment unit outputs experiment results which are transmitted to the data input unit through the data-path and transformed to independent variables. The data input unit and computing unit are communicated through the data-path; independent variables are transmitted to the computing unit through the data-path.

The independent variables include: values of C1, C2, C3, C4, C5;

preferably, said experiment results comprise: the number of ramA gene copies in the candidate Klebsiella strain, the number of sul1 gene copies in the candidate Klebsiella strain, the number of Klebsiella pneumoniae KPC-1 gene copies in the candidate Klebsiella strain, the number of DHA-1 gene copies in the candidate Klebsiella strain, the number of bleomycin resistance determinant gene copies in the candidate Klebsiella strain.

A method of predicting sensitivity of Klebsiella against Cefoxitin, characterized in that, comprising:

S1: k value is calculated according to formula I:

$$k = 0.032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) \qquad Formula I$$

$$k = 0. 32 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} )$$

S2: Exp(−k) power value with natural constant e as base and −k as exponent is calculated.

In formula I,

C1 is the number of ramA gene copies in a candidate Klebsiella strain,
C2 is the number of sul1 gene copies in a candidate Klebsiella strain,
C3 is the number of KPC-1 gene copies in a candidate Klebsiella strain,
C4 is the number of DHA-1 gene copies in a candidate Klebsiella strain,
C5 is the number of bleomycin resistance determinant gene copies in a candidate Klebsiella strain.

In above formula I, the first pair of parentheses includes C1 minus 1.008 divided by 0.317; the second pair of parentheses includes C2 minus 0.667 divided by 1.326; the third pair of parentheses includes C3 minus 0.552 divided by 1.121; the fourth pair of parentheses includes C4 minus 0.072 divided by 0.377; and the fifth pair of parentheses includes C5 minus 0.036 divided by 0.292.

In above formula I, k=0.032-0.557×(C1 minus 1.008 divided by 0.317)+0.054×(C2 minus 0.667 divided by 1.326)+2.878×(C3 minus 0.552 divided by 1.121)+1.021×(C4 minus 0.072 divided by 0.377)+0.772×(C5 minus 0.036 divided by 0.292).

A predicting result corresponding to Exp(−k) power value<1 is the candidate Klebsiella strain sensitive to Cefoxitin, and a predicting result corresponding to Exp(−k) power value≥1 is the candidate Klebsiella strain resistant against Cefoxitin.

said natural constant e=2.718281828459045.
the number of ramA, sul1, KPC-1, DHA-1, bleomycin resistance determinant gene copies in the candidate Klebsiella strain are obtained through a second generation high-throughput sequencing method.

The number of gene copies in the candidate Klebsiella strain=depth of gene contigs/depth of genome contigs;

preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depths of each contig which has said gene copies and said gene is located on;
preferably, each contig which has said gene copies is obtained by annotation of blat (v.36) software and diamond (v2.0.4.142) software through CARD database alignment between said gene cds and protein sequence;
preferably, depths of each contig which has said gene copies and said gene is located on are calculated through SPAdes v3.13.0 software.

One aspect of the present invention proposes a method of predicting sensitivity of Klebsiella against Cefoxitin.

In this invention, after routine processing of the obtained microbial samples, necessary steps such as DNA extraction and sequencing can be carried out. Through bioinformatics process analysis, the state of the relevant features of the Klebsiella prediction system in the samples can be obtained. The feature state information can be imported into the system to predict the drug sensitivity of the samples. Compared to traditional methods, it has advantages such as simple operation, short detection time, and accurate species identification.

In order to effectively evaluate the performance of a prediction system, it is necessary to establish a dataset that is not involved in the establishment of the prediction system, and evaluate the accuracy of the prediction system on this dataset. This independent dataset is called the test set. The evaluation methods for system prediction effectiveness include F1 score, Precision, Recall, and confusion matrix.

The method of the present invention also has the following advantages:

The present invention utilizes a test set to evaluate the accuracy of the system. The average accuracy of the method is 0.947, F1 score is 0.914, and recall score is 0.857. On the one hand, the present invention is less affected by subjective factors from such as operators, and has good detection stability; on the other hand, it achieves rapid and accurate identification of infectious pathogens and prediction of drug sensitivity of the test samples, meanwhile, it assists in diagnosis and rational & standardized medication treatment, has high throughput, and reduce medical costs.

### DESCRIPTION OF DRAWINGS

FIG. 1 is a schematic diagram of the structure of the drug resistance prediction system provided by some examples of the present invention (in dashed boxes) and its workflow diagram.

FIG. 2 is a schematic diagram of the structure of the drug resistance prediction system provided by other examples of the present invention (in dashed boxes) and its workflow diagram.

### EMBODIMENTS

In order to facilitate the understanding of the present invention, a more comprehensive description of the present invention will be provided in the embodiments below.

Unless otherwise defined, all technical and scientific terms used in this article have the same meanings as those commonly understood by a person skilled in the art of the present invention. The terms used in the specification of the present invention in this article are only for the purpose of describing specific embodiments and are not intended to limit the present invention.

The reagents used in the following embodiments are commercially available unless otherwise specified.

### Sources of Biomaterials

The 170 samples used in the experimental example of the present invention are pure cultures of Klebsiella strains isolated from clinical blood culture, from Beijing Union Medical College Hospital of the Chinese Academy of Medical Sciences.

All tested strains (strains) were identified as Klebsiella (scientific name: Genus Klebsiella, Latin name: Klebsiella Trevisan, systematic classification level: Genus) by mass spectrometry MALDI-TOF MS.

On the Illumina Novaseq NGS sequencing platform, these strains include 126 Klebsiella pneumoniae strains, 20 Klebsiella aerogenes strains, 8 Klebsiella oxytoca strains, 7 Klebsiella quasipneumoniae strains, 6 Klebsiella variicola strains, and 3 Klebsiella michiganensis strains, all of them are reported strains of the Klebsiella genus.

The above strains or strains can be obtained from common cases of Klebsiella pneumoniae pneumonia or from the applicant's laboratory. The applicant promises to distribute strains to the public within 20 years from the application date of the present invention for verifying the technical effects of the present invention.

### Examples Group 1. Resistant Predicting System of this Invention

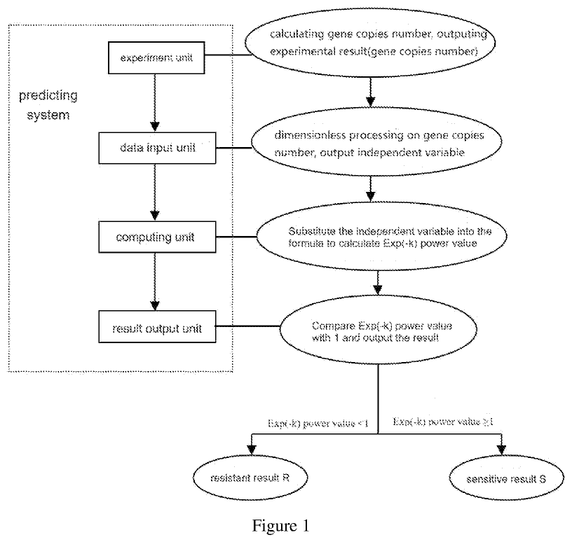

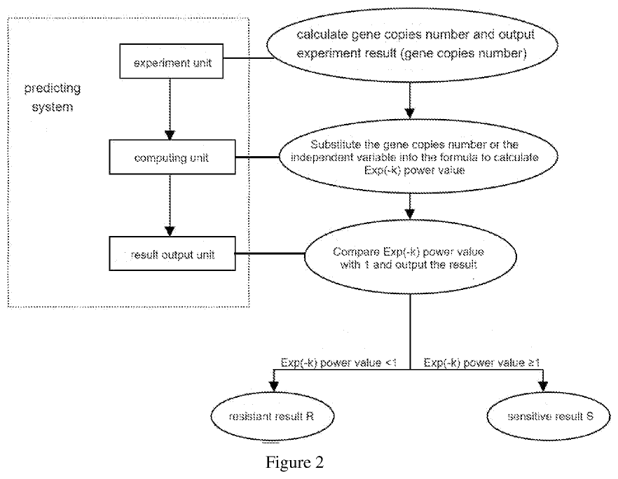

This group of examples provides a system of predicting sensitivity of Klebsiella against Cefoxitin. All examples of this group possess the following common features: as shown in FIG. 1 and FIG. 2, the system of predicting sensitivity of Klebsiella against Cefoxitin comprising: computing unit; said computing unit comprises: computer readable storage medium, which is stored with computer program, an Exp (−k) power value calculation method is implemented when said computer program is executed by a processor; said Exp(−k) power value calculation method comprises following computing steps:

S1: k value is calculated according to formula I:

$$k = 0.032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) \qquad Formula I$$

$$k = 0. 32 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} )$$

S2: Exp(−k) power value with natural constant e as base and −k as exponent is calculated;
in formula I,
C1 is the number of ramA gene copies in a candidate Klebsiella strain,
C2 is the number of sul1 gene copies in a candidate Klebsiella strain,
C3 is the number of KPC-1 gene copies in a candidate Klebsiella strain,
C4 is the number of DHA-1 gene copies in a candidate Klebsiella strain,
C5 is the number of bleomycin resistance determinant gene copies in a candidate Klebsiella strain.

In above formula I, the first pair of parentheses includes C1 minus 1.008 divided by 0.317; the second pair of parentheses includes C2 minus 0.667 divided by 1.326; the third pair of parentheses includes C3 minus 0.552 divided by 1.121; the fourth pair of parentheses includes C4 minus 0.072 divided by 0.377; and the fifth pair of parentheses includes C5 minus 0.036 divided by 0.292.

In above formula I, k=0.032−0.557×(C1 minus 1.008 divided by 0.317)+0.054×(C2 minus 0.667 divided by 1.326)+2.878×(C3 minus 0.552 divided by 1.121)+1.021×(C4 minus 0.072 divided by 0.377)+0.772×(C5 minus 0.036 divided by 0.292).

In some examples of this invention, said natural constant e=2.718281828459045.

In more specific examples, above genes are all known genes reported in the art.

ramA gene is ramA gene recorded in “Genetic regulation of the ramA locus and its expression in clinical isolates of Klebsiella pneumoniae”.

sul1 gene is sul1 gene recorded in “Co-occurrence of Klebsiella variicola and Klebsiella pneumoniae Both Carrying blaKPC from a Respiratory Intensive Care Unit Patient”.

KPC-1 gene is KPC-1 gene recorded in “Novel Carbapenem-Hydrolyzing b-Lactamase, KPC-1, from a Carbapenem-Resistant Strain of Klebsiella pneumoniae”.

DHA-1 gene is DHA-1 gene recorded in “Characterization of a DHA-1-Producing Klebsiella pneumoniae Strain Involved in an Outbreak and Role of the AmpR Regulator in Virulence”.

bleomycin resistance determinant gene is bleomycin resistance determinant gene recorded in “Association of the Emerging Carbapenemase NDM-1 with a Bleomycin Resistance Protein in Enterobacteriaceae and Acinetobacter baumannii”.

In further examples, as shown in FIG. 1 and FIG. 2, the system of predicting sensitivity of Klebsiella against Cefoxitin, also comprising: result output unit; the result output unit outputs sensitive result or resistant result; said sensitive result means the candidate Klebsiella strain is sensitive to Cefoxitin; said resistant result means the candidate Klebsiella strain is resistant against Cefoxitin;

the result output unit outputs resistant result R when Exp(−k) power value<1;
the result output unit outputs sensitive result S when Exp(−k) power value≥1;
preferably, the result output unit and the computing unit are communicated through data-path,
preferably, Exp(−k) power value calculated by the computing unit is transmitted to the result output unit through data-path;

In further examples, as shown in FIG. 1, the system of predicting sensitivity of Klebsiella against Cefoxitin, also comprising: experiment unit and data input unit;

the experiment unit and data input unit are communicated through data-path; the experiment unit outputs experiment results which are transmitted to the data input unit through data-path and transformed to independent variables;
said data input unit and computing unit are communicated through data-path; independent variables are transmitted to the computing unit through data-path.

In more specific example, said data-path is data transmission carrier well-known to a person skilled in the arts of computer and electronics. The data path can be selected from wired or wireless form, for example, it can be a wired path, line, wireless path, Wi-Fi connection, wireless channel, etc.

preferably, said independent variables include: values of C1, C2, C3, C4, C5;
preferably, said experiment results comprise: the number of ramA, sul1, KPC-1, DHA-1, bleomycin resistance determinant gene copies respectively in the candidate Klebsiella strain.

The copy number of known genes in known strains can be routinely obtained by a person skilled in the arts of molecular biology and bioinformatics through conventional techniques such as sequencing and bioinformatics analysis. The ramA, sul1, KPC-1, DHA-1, bleomycin resistance determinant genes involved in the experiment results output by the experiment unit of the prediction system of the present invention are all reported genes in the art, and their gene information and primary structural sequences can be queried through the NCBI website or other known bioinformatics databases. By conducting whole genome sequencing of the candidate Klebsiella strain, the numbers of each of the aforementioned genes copies in the strain can be obtained.

In other specific examples, the number of ramA, sul1, KPC-1, DHA-1, bleomycin resistance determinant gene copies in the candidate Klebsiella strain are obtained through a second-generation high-throughput sequencing method.

In more specific examples, the number of gene copies in the candidate Klebsiella strain=depth of gene contigs/depth of genome contigs;

preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depth of each contig which has said gene copies and said gene is located on;
preferably, each contig which has said gene copies is annotated by blat (v.36) software and diamond (v2.0.4.142) software through CARD database alignment between said gene cds and protein sequence;
preferably, depth of each contig which has said gene copies and said gene is located on are calculated through SPAdes v3.13.0 software.

The second-generation high-throughput sequencing method has a conventional technical meaning that is well-known to a person skilled in the art, while obtaining gene copy numbers using the second-generation high-throughput sequencing method is a conventional technical means that is well-known to a person skilled in the art.

In some specific embodiments, the specific method for calculating the gene copy number is as follows:

Sequencing of strains is conducted by using second-generation high-throughput sequencing methods. The average sequencing depth is about 150×, and the approximate sequencing amount for the Klebsiella genus is about 1G. Using the depth of contigs obtained and calculated by SPAdes (v3.13.0) assembly software during the assembly process as the standard, the longest contigs segment is defined as the genome segment. Prokka software (1.14.6) is used to conduct gene predicting on contigs, and all gene cds and protein sequences on contigs are obtained. blat (v.36) software and Diamond (v2.0.4.142) software are respectively used to compare the cds and protein sequences in the CARD database, sequences with a similarity greater than 90% are positive sequences, and annotation results for all resistance genes are obtained. The number of all gene copies on contigs are calculated using formula II as follows:

Formula II: the number of all gene copies on said contigs=depth of said contigs/depth of genome contigs

If a gene has two or more genome copies on different or the same contigs, the final number of gene copies is equal to the sum of all calculated number of the gene copies. Examples of calculation methods are as follows:

assuming that there is only one copy of the KPC-1 gene on all contigs, the number of the KPC-1 gene copies is:
the number of KPC-1 gene copies-depth of contigs which KPC-1 gene is located on/depth of genome contigs.

Assuming that the KPC-1 gene has 2 copies on one contigs and no copies on other contigs, the number of the KPC-1 gene copies is:

the number of KPC-1 gene copies-2×depth of contigs which KPC-1 gene is located on/depth of genome contigs.

Assuming that the KPC-1 gene has one copy respectively one contig1 and contig2, but no copies on other contigs, the number of the KPC-1 gene copies is:

the number of KPC-1 gene copies=depth of contig 1 which KPC-1 gene is located on/depth of genome contigs+depth of contig 2 which KPC-1 gene is located on/depth of genome contigs.

In more specific embodiments, the result output unit, experiment unit, and data input unit are all set as computer readable storage media on which computer programs are stored.

In some embodiments, when the computer program on the computer readable storage medium of the result output unit is executed by the processor, a method of comparing the Exp (−k) power value with 1 is implemented and the result is output;

That method of comparing the Exp (−k) power value with 1 is implemented and the result is output refers to:

the result output unit outputs resistant result R when Exp(−k) power value<1;
the result output unit outputs sensitive result S when Exp(−k) power value≥1;

In other embodiments, a method for calculating the number of gene copies is implemented when the computer program on the computer readable storage medium of the experiment unit is executed by the processor;

The method for calculating the number of gene copies is a conventional technical means well known to a person skilled in the art, and the specific steps are as follows:

S1: Take the maximum from depth of the genome contigs calculated by SPAdes v3.13.0 assembly software to obtain the genome contigs;
S2: Use BLAT (v.36) software and Diamond (v2.0.4.142) software to compare the CDs and protein sequences of a certain gene in the CARD database, and obtain each contigs with that gene copies by annotation;
S3: SPAdes v3.13.0 assembly software calculates the depth of the gene on each contigs has the gene copies;
S4: Calculate the sum of the depths of the gene on each contigs has the gene copies to obtain the depth of the contigs where the gene is located;
S5: Calculate the number of the gene copies according to the following formula, the number of copies=depth of gene contigs/depth of genome contigs.

In some embodiments, the computer program on the computer readable storage medium of the data input unit is executed by the processor to achieve dimensionless processing of the number of gene copies.

The dimensionless processing involves removing data dimensions or data units from the number of gene copies to obtain dimensionless values. Generally speaking, the data dimension or unit of the number of gene copies is: copy, number, or copies.

In other embodiments, as shown in FIG. 2, the system of predicting sensitivity of Klebsiella against meropenem may not require a data input unit. The experiment unit is directly connected to the computing unit through a data-path, allowing the number of gene copies (experiment results) or independent variable data calculated by the experiment unit to be directly input into the computing unit for Exp (−k) power value calculation.

### Examples Group 2. Predicting Method on Resistance of Klebsiella Against Cefoxitin of this Invention

This group of examples provides a method of predicting sensitivity of Klebsiella against Cefoxitin. All examples of this group possess the following common features: said method comprises:

S1: k value is calculated according to formula I:

$$k = 0.032 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) \qquad Formula I$$

$$k = 0. 32 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} )$$

S2: Exp(−k) power value with natural constant e as base and −k as exponent is calculated;
in formula I,
C1 is the number of ramA gene copies in a candidate Klebsiella strain,
C2 is the number of sul1 gene copies in a candidate Klebsiella strain,
C3 is the number of KPC-1 gene copies in a candidate Klebsiella strain,
C4 is the number of DHA-1 gene copies in a candidate Klebsiella strain,
C5 is the number of bleomycin resistance determinant gene copies in a candidate Klebsiella strain;
a predicting result corresponding to Exp(−k) power value<1 is the candidate Klebsiella strain sensitive to Cefoxitin, and a predicting result corresponding to Exp(−k) power value≥1 is the candidate Klebsiella strain resistant against Cefoxitin.

In above formula I, the first pair of parentheses includes C1 minus 1.008 divided by 0.317; the second pair of parentheses includes C2 minus 0.667 divided by 1.326; the third pair of parentheses includes C3 minus 0.552 divided by 1.121; the fourth pair of parentheses includes C4 minus 0.072 divided by 0.377; and the fifth pair of parentheses includes C5 minus 0.036 divided by 0.292.

In above formula I, k=0.032-0.557×(C1 minus 1.008 divided by 0.317)+0.054×(C2 minus 0.667 divided by 1.326)+2.878×(C3 minus 0.552 divided by 1.121)+1.021×(C4 minus 0.072 divided by 0.377)+0.772×(C5 minus 0.036 divided by 0.292).

In above formula I, e, as a mathematical constant, is the base of a natural logarithmic function, also known as a natural constant, natural base, or Euler number. It is an infinite non recurring decimal, which has the conventional technical meaning commonly understood by a ordinary technical person skilled in the art of mathematics. Its value is approximately: e=2.71828182845904523536.

In some examples of this invention, said natural constant e=2.718281828459045.

In some specific examples, the number of ramA, sul1, KPC-1, DHA-1, bleomycin resistance determinant gene copies in the candidate Klebsiella strain are obtained through a second generation high-throughput sequencing method.

In more specific examples, the number of gene copies in the candidate Klebsiella strain=depth of gene contigs/depth of genome contigs;

preferably, said genome contigs is a longest contigs segment assembled from sequencing results by SPAdes v3.13.0 software;
said depth of genome contigs is a depth of genome contigs calculated by SPAdes v3.13.0 software;
said depth of gene contigs refers to a sum of depths of each contig which has said gene copies and said gene is located on;
preferably, each contig which has said gene copies is obtained by annotation with blat (v.36) software and diamond (v2.0.4.142) software through CARD database alignment between said gene cds and protein sequence;
preferably, depths of each contigs which has said gene copies and said gene is located on are calculated through SPAdes v3.13.0 software.

The second-generation high-throughput sequencing method has a conventional technical meaning that is well-known to a person skilled in the art, while obtaining the number of gene copies by using the second-generation high-throughput sequencing method is a conventional technical means that is well-known to a person skilled in the art.

In some specific embodiments, the specific method for calculating the number of gene copies is as follows:

Sequencing of strains is conducted by using second-generation high-throughput sequencing methods. The average sequencing depth is about 150×, and the approximate sequencing amount for the Klebsiella genus is about 1G. Using the depth of contigs obtained and calculated by SPAdes (v3.13.0) assembly software during the assembly process as the standard, the longest contigs segment is defined as the genome segment. Prokka software (1.14.6) is used to conduct gene predicting on contigs, and all gene cds and protein sequences on contigs are obtained. blat (v.36) software and Diamond (v2.0.4.142) software are respectively used to compare the cds and protein sequences in the CARD database, sequences with a similarity greater than 90% are positive sequences, and annotation results for all resistance genes are obtained. The number of all gene copies on contigs are calculated using formula II as follows:

Formula II: the number of all gene copies on said contigs=depth of said contigs/depth of genome contigs

If a gene has two or more genome copies on different or the same contigs, the final number of gene copies is equal to the sum of all calculated number of the gene copies. Examples of calculation methods are as follows:

assuming that there is only one copy of the KPC-1 gene on all contigs, the number of the KPC-1 gene copies is:
the number of KPC-1 gene copies=depth of contigs which KPC-1 gene is located on/depth of genome contigs.

Assuming that the KPC-1 gene has 2 copies on one contigs and no copies on other contigs, the number of the KPC-1 gene copies is:

the number of KPC-1 gene copies=2× depth of contigs which KPC-1 gene is located on/depth of genome contigs.

Assuming that the KPC-1 gene has one copy respectively one contig1 and contig2, but no copies on other contigs, the number of the KPC-1 gene copies is:

the number of KPC-1 gene copies=depth of contig 1 which KPC-1 gene is located on/depth of genome contigs+depth of contig 2 which KPC-1 gene is located on/depth of genome contigs.

### Experimental Example. Performance Evaluation on Predicting System and Predicting Method of this Invention

The prediction system of the present invention was evaluated using 170 clinical samples, and the comparison between the broth micro dilution classification results and the system prediction results of 170 clinical samples is shown in Table 1. In the table below, S represents sensitivity and R represents resistance.

TABLE 1

| | | sensitive | Broth |
|---|---|---|---|
| sample | | predicting | microdilution |
| NO. | Exp(−k) | result | method result |
| s1 | 5.493506494 | S | S |
| s2 | 6.352941176 | S | S |
| s3 | 5.578947368 | S | S |
| s4 | 5.451612903 | S | S |
| s5 | 6.352941176 | S | S |
| s6 | 6.194244604 | S | S |
| s7 | 0.02145046 | R | R |
| s8 | 5.451612903 | S | S |
| s9 | 0.116071429 | R | R |
| s10 | 0.038421599 | R | R |
| s11 | 5.493506494 | S | S |
| s12 | 4.025125628 | S | R |
| s13 | 6.633587786 | S | S |
| s14 | 6.299270073 | S | S |
| s15 | 0.023541453 | R | R |
| s16 | 4.154639175 | S | S |
| s17 | 5.493506494 | S | S |
| s18 | 6.042253521 | S | S |
| s19 | 5.578947368 | S | S |
| s20 | 4 | S | S |
| s21 | 5.578947368 | S | S |
| s22 | 0.017293998 | R | R |
| s23 | 5.25 | S | S |
| s24 | 5.024096386 | S | S |
| s25 | 5.211180124 | S | S |
| s26 | 0.072961373 | R | R |
| s27 | 4.813953488 | S | S |
| s28 | 6.407407407 | S | S |
| s29 | 0.019367992 | R | R |
| s30 | 5.329113924 | S | S |
| s31 | 14.38461538 | S | R |
| s32 | 6.246376812 | S | S |
| s33 | 0.035196687 | R | R |
| s34 | 6.246376812 | S | S |
| s35 | 5.993006993 | S | S |
| s36 | 0.084598698 | R | R |
| s37 | 5.369426752 | S | S |
| s38 | 6.194244604 | S | S |
| s39 | 0.046025105 | R | R |
| s40 | 5.41025641 | S | S |
| s41 | 5.535947712 | S | S |
| s42 | 0.020408163 | R | R |
| s43 | 5.211180124 | S | S |
| s44 | 0.049317943 | R | R |
| s45 | 4.291005291 | S | S |
| s46 | 6.352941176 | S | S |
| s47 | 0.022494888 | R | R |
| s48 | 0.016260163 | R | R |
| s49 | 5.211180124 | S | S |
| s50 | 6.042253521 | S | S |
| s51 | 6.142857143 | S | S |
| s52 | 4.464480874 | S | S |
| s53 | 0.002004008 | R | R |
| s54 | 2.058103976 | S | R |
| s55 | 4.524861878 | S | S |
| s56 | 6.462686567 | S | S |
| s57 | 0.042752868 | R | R |
| s58 | 6.042253521 | S | S |
| s59 | 6.246376812 | S | S |
| s60 | 5.802721088 | S | S |
| s61 | 6.633587786 | S | S |
| s62 | 5.25 | S | S |
| s63 | 0.014198783 | R | R |
| s64 | 5.289308176 | S | S |
| s65 | 0.062699256 | R | R |
| s66 | 6.092198582 | S | S |
| s67 | 5.451612903 | S | S |
| s68 | 0.035196687 | R | R |
| s69 | 1.02020202 | S | S |
| s70 | 5.849315068 | S | S |
| s71 | 4.952380952 | S | S |
| s72 | 5.41025641 | S | S |
| s73 | 4.714285714 | S | S |
| s74 | 0.960784314 | R | S |
| s75 | 0.189060642 | R | R |
| s76 | 5.802721088 | S | S |
| s77 | 0.070663812 | R | R |
| s78 | 4.952380952 | S | S |
| s79 | 0.017293998 | R | R |
| s80 | 4.847953216 | S | R |
| s81 | 0.018329939 | R | R |
| s82 | 5.802721088 | S | S |
| s83 | 0.015228426 | R | R |
| s84 | 5.25 | S | S |
| s85 | 4.649717514 | S | R |
| s86 | 5.578947368 | S | S |
| s87 | 4.882352941 | S | S |
| s88 | 1.02020202 | S | S |
| s89 | 5.666666667 | S | S |
| s90 | 4.347593583 | S | S |
| s91 | 5.493506494 | S | S |
| s92 | 5.451612903 | S | S |
| s93 | 5.172839506 | S | S |
| s94 | 5.451612903 | S | S |
| s95 | 5.329113924 | S | S |
| s96 | 6.299270073 | S | S |
| s97 | 0.001001001 | R | R |
| s98 | 0 | R | R |
| s99 | 5.493506494 | S | S |
| s100 | 5.211180124 | S | S |
| s101 | 0.157407407 | R | R |
| s102 | 5.711409396 | S | S |
| s103 | 5.711409396 | S | S |
| s104 | 0.023541453 | R | R |
| s105 | 0.026694045 | R | R |
| s106 | 6.751937984 | S | R |
| s107 | 0.074113856 | R | R |
| s108 | 5.622516556 | S | S |
| s109 | 6.092198582 | S | S |
| s110 | 6.352941176 | S | S |
| s111 | 5.097560976 | S | S |
| s112 | 0.051524711 | R | R |
| s113 | 6.194244604 | S | S |
| s114 | 0.083423619 | R | R |
| s115 | 5.666666667 | S | S |
| s116 | 5.535947712 | S | S |
| s117 | 6.462686567 | S | S |
| s118 | 5.369426752 | S | S |
| s119 | 4.681818182 | S | S |
| s120 | 0.004016064 | R | R |
| s121 | 6.575757576 | S | S |
| s122 | 0.042752868 | R | R |
| s123 | 5.756756757 | S | S |
| s124 | 5.711409396 | S | S |
| s125 | 5.451612903 | S | S |
| s126 | 0.052631579 | R | R |
| s127 | 0.002004008 | R | R |
| s128 | 5.756756757 | S | S |
| s129 | 6.407407407 | S | S |
| s130 | 4.747126437 | S | R |
| s131 | 5.622516556 | S | S |
| s132 | 4.917159763 | S | S |
| s133 | 0.025641026 | R | R |
| s134 | 0.018329939 | R | R |
| s135 | 6.142857143 | S | S |
| s136 | 5.369426752 | S | S |
| s137 | 5.802721088 | S | S |
| s138 | 4.988023952 | S | R |
| s139 | 0.088139282 | R | R |
| s140 | 6.633587786 | S | S |
| s141 | 4.681818182 | S | S |
| s142 | 4.586592179 | S | S |
| s143 | 5.024096386 | S | S |
| s144 | 5.451612903 | S | S |
| s145 | 4.714285714 | S | S |
| s146 | 5.493506494 | S | S |
| s147 | 0.008064516 | R | R |
| s148 | 0.023541453 | R | R |
| s149 | 0.001001001 | R | R |
| s150 | 0.012145749 | R | R |
| s151 | 5.756756757 | S | S |
| s152 | 6.042253521 | S | S |
| s153 | 0.016260163 | R | R |
| s154 | 5.666666667 | S | S |
| s155 | 7.130081301 | S | S |
| s156 | 5.211180124 | S | S |
| s157 | 5.802721088 | S | S |
| s158 | 0.023541453 | R | R |
| s159 | 5.41025641 | S | S |
| s160 | 0.006036217 | R | R |
| s163 | 5.993006993 | S | S |
| s162 | 5.329113924 | S | S |
| s163 | 5.622516556 | S | S |
| s164 | 5.622516556 | S | S |
| s165 | 0 | R | R |
| s166 | 0.040582726 | R | R |
| s167 | 4.494605495 | S | S |
| s168 | 5.25 | S | S |
| s169 | 5.944444444 | S | S |
| s170 | 5.493506494 | S | S |

The confusion matrix generated by the test result data is shown in Table 2:

TABLE 2

| | | predicting result | |
|---|---|---|---|
| confusion matrix | | R | S |
| real result | R | 48 | 8 |
| | S | 1 | 113 |

Assuming TP (True Positive) represents the number of true positive cases, FP (False Positive) represents the number of false positive cases, FN (False Negative) represents the number of false negative cases, and TN (Ture Negative) represents the number of true negative cases. Precision refers to the proportion of positive samples in the positive case determined by the classifier. The recall rate refers to the proportion of predicted positive cases to the total positive cases. Accuracy refers to the proportion of correct judgments made by the classifier on the entire sample. F1 score is the harmonic mean of accuracy and recall, with a maximum of 1 and a minimum of 0. The calculation results of each indicator are as follows:

$$precision = \frac{TP}{TP + FP} = \frac{48}{48 + 1} = 0.98$$

$$recall = \frac{TP}{TP + FN} = \frac{48}{48 + 8} = 0.857$$

$$accuracy = \frac{TP + TN}{TP + FP + TN + FN} = \frac{48 + 113}{48 + 1 + 113 + 8} = 0.947$$

$$F 1 = \frac{2 \times precision \times recall}{precision + recall} = 0.914$$

The above examples only express the embodiments of the present invention, and their description is more specific and detailed, but they cannot be understood as a limitation on the scope of the invention patent. It should be pointed out that for an ordinary technical person skilled in the art, several deformations and improvements can be made without departing from the concept of the present invention, all of which fall within the scope of protection of the present invention. Therefore, the scope of protection of the present invention patent should be based on the claims.

1.-8. (canceled)

9. A method of predicting sensitivity of a candidate Klebsiella strain against Cefoxitin, comprising:
obtaining a number of ramA gene copies in the candidate Klebsiella strain, a number of sul1 gene copies in the candidate Klebsiella strain, a number of KPC-1 gene copies in the candidate Klebsiella strain, a number of DHA-1 gene copies in the candidate Klebsiella strain, and a number of bleomycin resistance determinant gene copies in the candidate Klebsiella strain;
calculating a value k according to formula I:
$$k = 0.03 2 - 0.557 \times ( \frac{C 1 - 1.008}{0.317} ) + 0.054 \times ( \frac{C 2 - 0.667}{1.326} ) + 2.878 \times ( \frac{C 3 - 0.552}{1.121} ) + 1.021 \times ( \frac{C 4 - 0.072}{0.377} ) + 0.772 \times ( \frac{C 5 - 0.036}{0.292} ) ; \qquad Formula I$$
calculating an Exp(−k) power value with a natural constant e as a base and −k as an exponent, wherein Exp(−k)=e^{-k}; and
predicting sensitivity of the candidate Klebsiella strain against Cefoxitin, wherein when the Exp(−k) power value is less than 1, the candidate Klebsiella strain is sensitive to Cefoxitin and when the Exp(−k) power value is greater than or equal to 1, the candidate Klebsiella strain is resistant against Cefoxitin,
wherein
C1 is the number of ramA gene copies in a candidate Klebsiella strain,
C2 is the number of sul1 gene copies in a candidate Klebsiella strain,
C3 is the number of KPC-1 gene copies in a candidate Klebsiella strain,
C4 is the number of DHA-1 gene copies in a candidate Klebsiella strain, and
C5 is the number of bleomycin resistance determinant gene copies in a candidate Klebsiella strain.

10. The method of predicting sensitivity of the candidate Klebsiella strain against Cefoxitin according to claim 9, wherein said natural constant e=2.718281828459045.

11. The method of predicting sensitivity of the candidate Klebsiella strain against Cefoxitin according to claim 9, wherein the number of ramA, sul1, KPC-1, DHA-1, and bleomycin resistance determinant gene copies in the candidate Klebsiella strain are obtained through a second generation high-throughput sequencing method.

12. The method of predicting sensitivity of the candidate Klebsiella strain against Cefoxitin according to claim 11, wherein the number of gene copies in the candidate Klebsiella strain is equal to a depth of gene contigs divided by a depth of genome contigs.

13. The method of predicting sensitivity of the candidate Klebsiella strain against Cefoxitin according to claim 12, further comprising:
assembling said genome contigs from sequencing results to generate a longest contigs segment; and
calculating said depth of genome contigs;
wherein said depth of gene contigs refers to a sum of depths of each contig which has said gene copies and said gene is located on.

14. The method of predicting sensitivity of the candidate Klebsiella strain against Cefoxitin according to claim 13, wherein each contig which has said gene copies is annotated through a comprehensive antibiotic resistance database (CARD) alignment between gene cds and protein sequences.

15. The method of predicting sensitivity of the candidate Klebsiella strain against Cefoxitin according to claim 13, wherein depths of each contig which has said gene copies and said gene is located on are calculated through assembly software.

16. The method of predicting sensitivity of the candidate Klebsiella strain against Cefoxitin according to claim 9, wherein obtaining includes calculating the numbers of gene copies by, for each gene type:
obtaining contigs having gene copies of the gene type by comparing coding sequences and protein sequences of genes in a database,
calculating a depth of the gene type on each contig having gene copies of the gene type,
calculating a sum of depths of the gene type on each contig having the gene type to obtain a depth of contigs where the gene type is located, and
calculating the number of the gene copies by dividing the depth of the gene contigs by the depth of the genome contigs.

17. The method of predicting sensitivity of the candidate Klebsiella strain against Cefoxitin according to claim 9, wherein obtaining numbers of gene copies includes conducting whole genome sequencing of the candidate Klebsiella strain.

18. The method of predicting sensitivity of the candidate Klebsiella strain against Cefoxitin according to claim 9, wherein obtaining numbers of gene copies includes querying a bioinformatics database for gene information and primary structural sequences.
